(19) United States
(12) Patent Application Publication
(10) Pub. No.: US 2023/0238121 A1
(43) Pub. Date: Jul. 27, 2023

(54) EQUIPMENT UTILIZATION AND HEALTHCARE TECHNOLOGY MANAGEMENT PLATFORM

(71) Applicant: SQUID iQ, Inc., Altamonte Springs, FL (US)

(72) Inventor: Robert DAVENPORT, San Francisco, CA (US)

(21) Appl. No.: 18/101,035

(22) Filed: Jan. 24, 2023

Related U.S. Application Data
(60) Provisional application No. 63/302,975, filed on Jan. 25, 2022.

(51) Int. Cl. G16H 40/20

(52) U.S. Cl. CPC G16H 40/20

(57) ABSTRACT

A method for generating and displaying a user interface for managing medical devices utilized in a medical facility is provided. The method includes accessing device data and service data associated with a plurality of medical devices utilized in at least one medical facility and integrating the device data and service data to generate an integrated device and service dataset. The method further includes determining, based on the integrated device and service dataset, a tracking identification and a cost value of each of the plurality of medical devices, determining performance or usage metrics of each of the plurality of medical devices based on the tracking identification and the cost value, and causing, based on the performance or usage metrics, a user interface (UI) executing on electronic devices to display a visual representation of the performance or usage metrics.

### CROSS-REFERENCE TO RELATED APPLICATIONS

This application claims the benefit under 35 U.S.C. § 119(e) of U.S. Provisional Patent Application No. 63/302,975, filed Jan. 25, 2022, which is incorporated herein by reference.

### TECHNICAL FIELD

This application relates generally to healthcare technology management, and, more particularly, to an equipment utilization and healthcare technology management platform and user interface.

### BACKGROUND

Healthcare technology management (HTM) systems may generally include systems for managing the selection, maintenance, and safe use of medical devices and equipment that may be utilized in a medical facility. In some instances, the HTM systems, and the associated medical devices and equipment, may be maintained by one or more local biomedical technicians interfacing with a number of remote manufacturers across a large and unrelated ecosystem of medical devices and equipment that may be included within any number of medical facilities. However, existing HTM systems may lack any data or inferences relating to the specific performance, usage, or costs of each individual medical device and equipment that may be deployed in any particular medical facility.

### SUMMARY

Embodiments of the present disclosure are directed toward one or more computing devices, methods, and non-transitory computer-readable media for generating and displaying a user interface for managing medical devices utilized in a medical facility. In certain embodiments, the one or more computing devices may access device data and service data associated with a plurality of medical devices utilized in at least one medical facility. In some embodiments, the device data may include a device identification data obtained from a manufacturer of each of the plurality of medical devices or a regulatory agency associated with each of the plurality of medical devices. In some embodiments, the service data may include transaction data obtained from one or more of a purchase contract associated with each of the plurality of medical devices, a service contract associated with each of the plurality of medical devices, or an enterprise resource planning (ERP) system associated with each of the plurality of medical devices. In certain embodiments, the one or more computing devices may then integrate the device data and service data to generate an integrated device and service dataset associated with the plurality of medical devices. In certain embodiments, the one or more computing devices may then determine, based on the integrated device and service dataset, a tracking identification and a cost value of each of the plurality of medical devices.

In certain embodiments, the one or more computing devices may then determine, based on the tracking identification and the cost value, one or more performance or usage metrics of each of the plurality of medical devices. In some embodiments, the one or more performance or usage metrics may include one or more of a utilization, a location, a capacity, a status, an asset aging, a cost basis, a maintenance cost, a maintenance utilization, a maintenance performance, a vendor performance, a clinical unit statistic, or a device performance. In certain embodiments, the one or more computing devices may estimate the one or more performance or usage metrics by estimating by an asset utilization of each of the plurality of medical devices or a total cost of ownership of each of the plurality of medical devices. In certain embodiments, the one or more computing devices may then cause, based on the one or more performance or usage metrics, a user interface (UI) executing on one or more electronic devices to display a visual representation of the one or more performance or usage metrics.

In some embodiments, the visual representation of the one or more performance or usage metrics may include a real-time or near real-time dashboard associated with the plurality of medical devices utilized in the at least one medical facility. In some embodiments, the one or more computing devices may receive one or more user inputs from one or more of the plurality of medical devices, the one or more user inputs being performed on the one or more of the plurality of medical devices by a clinician in at least one medical facility. In some embodiments, the one or more computing devices may update the UI based on the one or more user inputs.

### BRIEF DESCRIPTION OF THE DRAWINGS

FIG. 1A illustrates an example embodiment of an equipment utilization and healthcare technology management platform.

FIG. 1B illustrates example user interfaces of an equipment utilization and healthcare technology management platform.

FIG. 2 illustrates a detailed embodiment of an equipment utilization and healthcare technology management platform and medical facility client system.

FIG. 3 illustrates an example embodiment of a dashboard for viewing and managing medical devices utilized in a medical facility.

FIG. 4A illustrates a flow diagram of a method for estimating performance or usage metrics of medical devices utilized within a medical facility.

FIG. 4B illustrates a flow diagram of a method for generating and displaying a user interface for managing medical devices utilized in a medical facility.

FIG. 5 illustrates an example computing system.

### DESCRIPTION OF EXAMPLE EMBODIMENTS

Embodiments of the present disclosure include a platform that can be used by both medical practitioners (e.g., doctors, nurses, bio-medical engineers) and administrators (e.g., hospital assets administrators). Medical practitioners may use the platform to obtain location data of one or more assets (e.g., GPS coordinates, room in the hospital, etc.) and availability data of the one or more assets (e.g., whether an asset can be claimed for use). In some embodiments, the location data and/or the availability data can be provided in a real-time or near-real-time manner on a user device such that medical practitioners can understand whether an asset of interest is available for use and where to obtain the asset of interest. Further, administrators may use the platform to obtain a variety of information related to the management and utilization of one or more assets, such as the quantity of an asset, the utilization rate of the asset (e.g., how many units of the asset are currently claimed for use or in use), cost of ownership of the asset, etc.

The exemplary platform can provide a combination of data integration processes and automated workflows to provide the functionalities described herein. In some embodiments, the platform combines data integration with workflow automation to uniquely provide the visibility and manageability of medical (or other specialized) equipment in high-stakes, real-time operating environments.

For example, the platform can leverage data integration processes to obtain and integrate data from multiple, disparate data sources. While such data may include various discrepancies and be fragmented, the exemplary platform can integrate and process such data in the repository for downstream use.

Further, the exemplary platform can provide automated workflows using one or more user interfaces available on user devices (e.g., mobile phones). For example, a medical practitioner may access the platform to search for an asset of interest to find location data and availability data associated with various units of the asset. The available asset(s) or units of asset(s) may be displayed in a list in the order of closest proximity. The medical practitioner can use the platform to claim an asset for use in the inventory, dispatch a person to obtain the asset, or a combination thereof.

Accordingly, embodiments of the present disclosure can automate equipment/asset utilization and management processes. Traditionally, the tracking of equipment utilization is manual, inefficient, and error-prone. The platform can help various user groups to find what they need, track location data and availability data, claim assets for use, and understand the status of the entire inventory in a precise and real-time manner. Accordingly, the platform can optimize utilization of assets and minimize logistical burden, thus saving time and resources for the organization and reducing the need to acquire external assets (e.g., via renting). The platform can enable process assurance and financial control of expenses and costs related to provision of medical equipment capacity and utilization.

It should be appreciated that the techniques described herein are not limited to management of hospital and medical equipment. In some embodiments, the techniques can be used in other organizations for managing assets, such as mining/energy processing plants, etc.

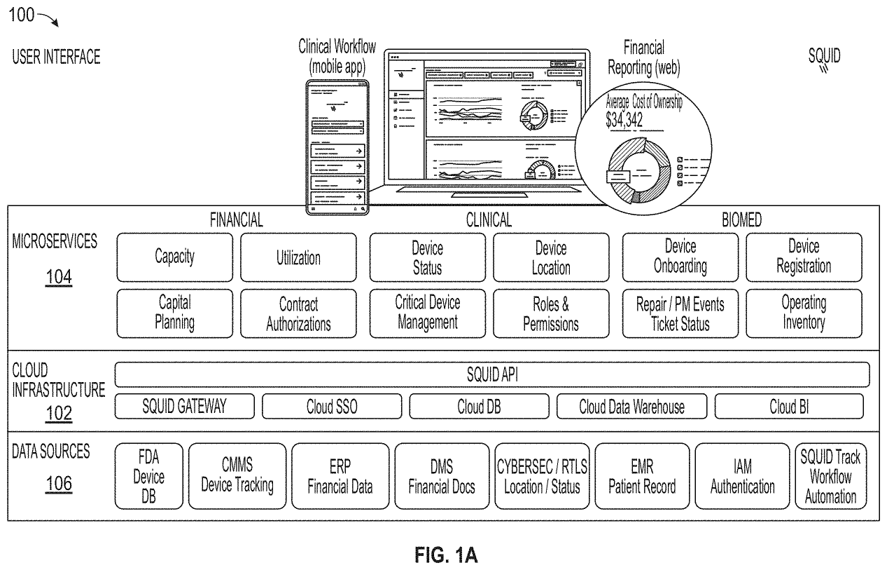

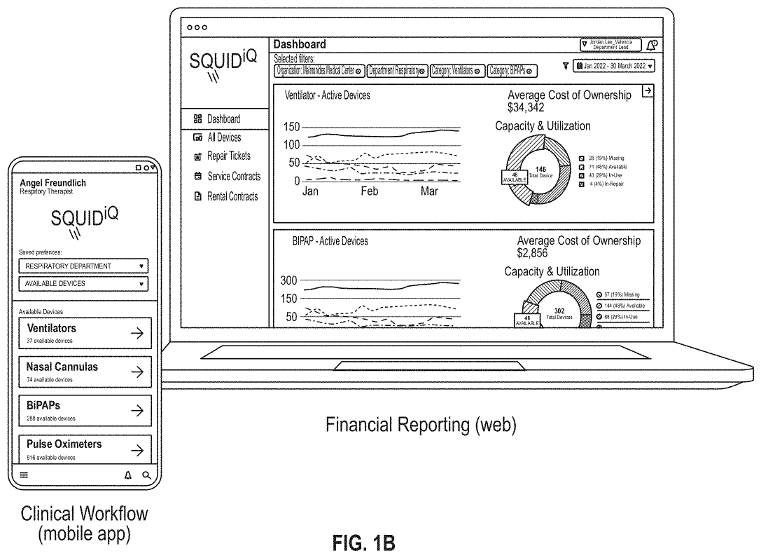

FIG. 1A illustrates an example embodiment of an equipment utilization and healthcare technology management platform 100 that may be utilized for estimating performance or usage metrics of medical devices utilized within a medical facility and generating and displaying a user interface for managing medical devices utilized in the medical facility, in accordance with the presently disclosed embodiments. As depicted, in certain embodiments, the equipment utilization management platform 100 may include a cloud-based infrastructure 102, which may receive various data from a number of primary data sources 104. In certain embodiments, the cloud-based infrastructure 102 may be utilized to estimate performance or usage metrics of medical devices utilized within a medical facility (e.g., hospital, clinic, care center, specialized treatment center, and so forth) and to generate and display a real-time or near real-time location, status, and utilization data through a mobile and/or web-based user interface (UI) dashboard for managing medical devices utilized in the medical facility. For example, as further depicted by FIG. 1A, the cloud-based infrastructure 102 may further utilize the estimated performance or usage metrics of medical devices to perform or generate a number of microservices 106. FIG. 1B illustrates example user interfaces of the equipment utilization and healthcare technology management platform.

In certain embodiments, the cloud-based infrastructure 102 may include a Platform as a Service (PaaS), a Software as a Service (SaaS), a Compute as a Service (CaaS), an Infrastructure as a Service (IaaS), a Data as a Service (DaaS), a Database as a Service (DBaaS), or other similar cloud-based computing architecture (e.g., “X” as a Service (XaaS)) that may be suitable for estimating performance or usage metrics of medical devices and generating and displaying a real-time or near real-time dashboard for managing the medical devices in accordance with the presently disclosed embodiments. In certain embodiments, as further depicted by FIG. 1A, the number of primary data sources 104 may include one or more of a hospital or clinical biomedical engineering data source that may be accessed by way of a computerized maintenance management system (CMMS) application programming interface (API), one or more internet-of-things (IoT) data source including a log of an ecosystem of medical devices that may be utilized within a medical facility (e.g., hospital, clinic, care center, specialized treatment center, and so forth) and accessible by way of a cyber-security API, a real-time location system (RTLS) for keeping track of the medical devices throughout the medical facility, a regulatory agency data source including unique device identification (UDI) data accessible by way of a UDI API, critical device management (CDM) data source including clinical data associated with the medical facility that may be accessible by way of a CDM API, a financials and transactions data source including the acquisition costs, maintenance costs, or service costs of each of the medical devices accessible by way of an enterprise resource planning (ERP) API, and a purchase contracts and service contracts data source including various transactions, services, and contracts associated with each of the medical devices accessible by way of a contract management API.

In certain embodiments, as further depicted by FIG. 1A, the number of microservices 106 that may be performed or generated by the cloud-based infrastructure 102 may include medical device management and analytics, managing department medical device inventory, managing and keeping track of medical device location, analyzing service contracts, and generating and managing maintenance and repair tickets for each of the medical devices.

In certain embodiments, the cloud-based infrastructure 102 may be utilized to ascertain user requirements and workflows related to equipment use, then determine how best to source and report on the equipment capacity required which entails integrating relevant data from various sources and devices to incorporate financial, maintenance, utilization and device information and providing in real-time or near real-time to user groups across a particular medical facility (e.g., hospital, clinic, care center, specialized treatment center, and so forth). This may be combined with workflow automation dashboard for clinicians who use the equipment to provide services to augment the visibility and manageability of the equipment inventory. Thus, the cloud-based infrastructure 102 may enable a workflow automation dashboard displaying a 360-degree view of equipment capacity, utilization, location, status and total cost of ownership.

In certain embodiments, the cloud-based infrastructure 102 mediates ingestion, transformation, encryption and reporting. For example, the one or more server gateway modules may include an edge encryption mechanism that allows privileged information to be incorporated into user interfaces and dashboards without requiring any storage of client privileged information to the cloud infrastructure 102. The mobile user interface enables workflow automation (e.g., saving users time, ensuring process adherence, and facilitating process improvement); utilization, location, and status reporting; and utilization event data capture. In certain embodiments, RTLS may be implemented in association with the cloud-based infrastructure 102 using standard Wi-Fi/Bluetooth Low Energy (BLE) networks, mobile phones, or other mechanisms to determine user location and relative locations of users to specific devices they need in order of proximity to the user. In some embodiments, the cloud-based infrastructure 102 may be context, network, information system, and device agnostic and may be implemented incrementally and efficiently. In one embodiment, the cloud-based infrastructure 102 may include a learning platform that better anticipates user needs over time, rendering the cloud-based infrastructure 102 increasingly responsive with less user intervention over time.

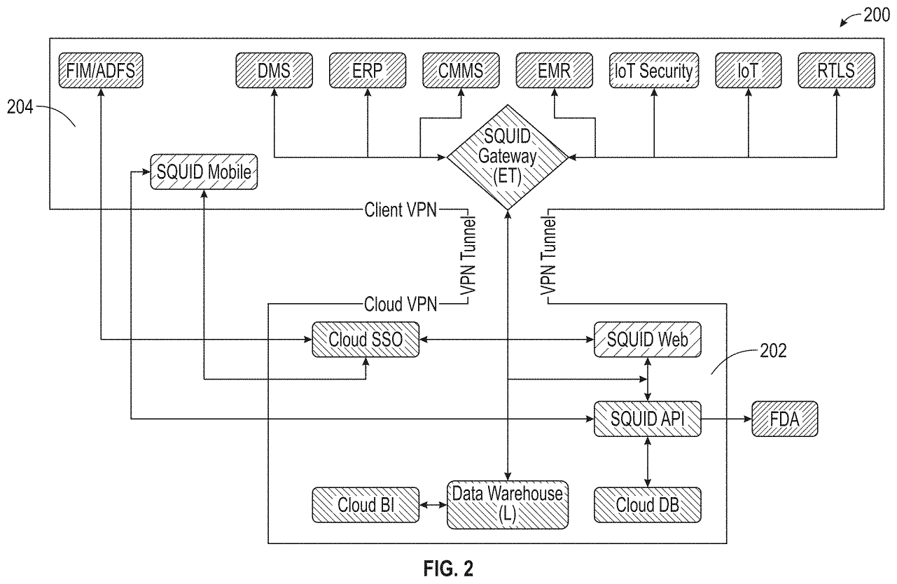

FIG. 2 illustrates a detail embodiment of an equipment utilization and healthcare technology management platform and medical facility client system 200 that may be utilized for estimating performance or usage metrics of medical devices utilized within a medical facility and generating and displaying a user interface for managing medical devices utilized in the medical facility, in accordance with the presently disclosed embodiments. As depicted, the equipment utilization and healthcare technology management platform and medical facility client system 200 may include an equipment utilization and healthcare technology management platform 202 and a medical facility client system 204. In one embodiment, the equipment utilization and healthcare technology management platform 202 may correspond to the cloud-based infrastructure 102 as discussed above with respect to FIG. 1A. Similarly, the medical facility client system 204 may collectively correspond to the number of primary data sources 104 discussed above with respect to FIG. 1A.

In certain embodiments, one or more client gateway modules of the medical facility client system 204 may receive device data and service data from the number of primary data sources 104. The one or more client gateway modules of the medical facility client system 204 may then pass the received device data and service data to one or more server gateway modules of the equipment utilization and healthcare technology management platform 202. In certain embodiments, the one or more server gateway modules of the equipment utilization and healthcare technology management platform 202 may then clean, correlate, and integrate the device data and the service data to generate a correlated and integrated device and service dataset associated with each of the number of medical devices.

In certain embodiments, the one or more server gateway modules in conjunction with one or more events modules and relational databases of the equipment utilization and healthcare technology management platform 202, may then determine, based on the correlated and integrated device and service dataset, a tracking identification and a cost value of each of the number of medical devices. For example, in one embodiment, the tracking identification may include one or more unique device identifiers (UDIs) that may be utilized to identify and track each of the medical devices over the course of the respective lifecycles of each of the number of medical devices. Similarly, in some embodiments, the cost value may include any of various acquisition costs, maintenance costs, or service costs associated with each of the number of medical devices as incurred, for example, by a particular medical facility (e.g., hospital, clinic, care center, specialized treatment center, and so forth).

In certain embodiments, the one or more server gateway modules in conjunction with one or more events modules and relational databases of the equipment utilization and healthcare technology management platform 202 may then estimate, based on the tracking identification and the cost value, one or more performance or usage metrics associated with each of the number of medical devices utilized in the particular medical facility (e.g., hospital, clinic, care center, specialized treatment center, and so forth). In one embodiment, the one or more server gateway modules of the equipment utilization and healthcare technology management platform 202 may utilize one or more machine-learning models to estimate the one or more performance or usage metrics associated with each of the number of medical devices utilized in the particular medical facility (e.g., hospital, clinic, care center, specialized treatment center, and so forth). In some embodiments, the one or more performance or usage metrics may include one or more of a utilization, a location, a capacity, a status, an asset aging, a cost basis, a maintenance cost, a maintenance utilization, a maintenance performance, a vendor performance, a clinical unit statistic, or a device performance for each of the number of medical devices utilized in the particular medical facility (e.g., hospital, clinic, care center, specialized treatment center, and so forth).

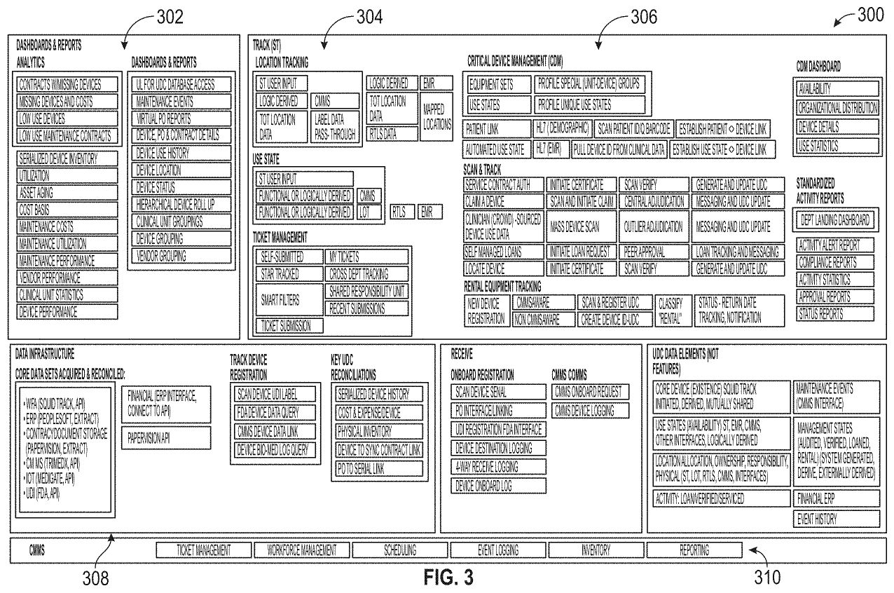

In certain embodiments, the one or more server gateway modules in conjunction with one or more events modules and relational databases of the equipment utilization and healthcare technology management platform 202 may also estimate an asset utilization of each of the number of medical devices and a total cost of ownership of each of the number of medical devices as utilized in the particular medical facility (e.g., hospital, clinic, care center, specialized treatment center, and so forth). In certain embodiments, as will be further illustrated by FIG. 3 below, the one or more server gateway modules in conjunction with one or more events modules and relational databases of the equipment utilization and healthcare technology management platform 202 may then generate and caused to be displayed a real-time or near real-time dashboard for managing the number of medical devices as utilized in the particular medical facility (e.g., hospital, clinic, care center, specialized treatment center, and so forth).

FIG. 3 illustrates an example embodiment of a user interface 300 (e.g., real-time or near real-time dashboard) for viewing and managing medical devices utilized in a medical facility, in accordance with the presently disclosed embodiments. As depicted, in certain embodiments, the user interface 300 (e.g., real-time or near real-time dashboard) may include dashboard and reports 302, location tracking log 304, CDM system and dashboard 306, financial data infrastructure log 308, and application display tabs 310. As depicted, the dashboard and reports 302 may include various medical device analytics data including, for example, contracts with missing devices, missing medical devices and costs, low use medical devices, low use maintenance contracts, medical device inventory, medical device utilization, medical device asset aging, cost basis, maintenance costs, maintenance utilization, maintenance performance, vendor performance, clinical unit statistics, equipment status, and medical device performance.

In certain embodiments, the location tracking log 304 may include medical device location tracking data for each of a number of medical devices as utilized in a particular medical facility (e.g., hospital, clinic, care center, specialized treatment center, and so forth), use state or status for each of the number of medical devices as utilized in the particular medical facility (e.g., hospital, clinic, care center, specialized treatment center, and so forth), and maintenance or service tickets for each of the number of medical devices as utilized in the particular medical facility (e.g., hospital, clinic, care center, specialized treatment center, and so forth). Similarly, the CDM system and dashboard 306 may include logs of each of a number of critical medical devices (e.g., which may include a specific subset of the total number of medical devices utilized in a particular medical facility) as these medical devices are utilized, rented, or borrowed by clinicians within the particular medical facility (e.g., hospital, clinic, care center, specialized treatment center, and so forth). The financial data infrastructure log 308 may include a log of data relating to the capitalization, asset utilization, or total cost of ownership of each of the number of medical devices as utilized in the particular medical facility (e.g., hospital, clinic, care center, specialized treatment center, and so forth). It also includes which equipment is available for use, in repair, missing, and when known, where it is. The application display tabs 310 may include a number of user-selectable tabs that may display additional information for each of the number of medical devices as utilized in the particular medical facility (e.g., hospital, clinic, care center, specialized treatment center, and so forth).

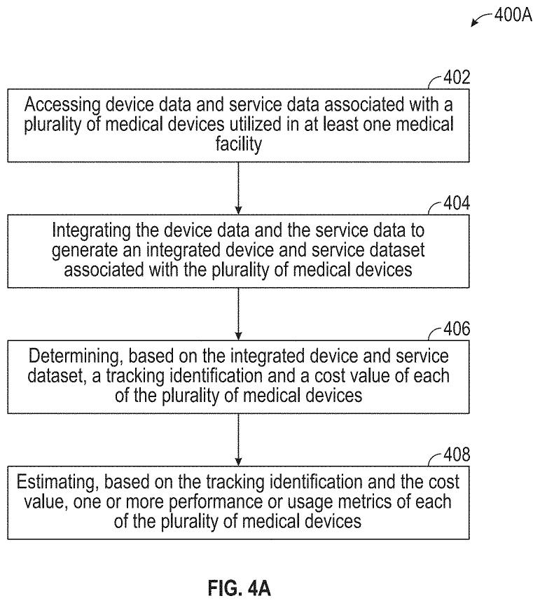

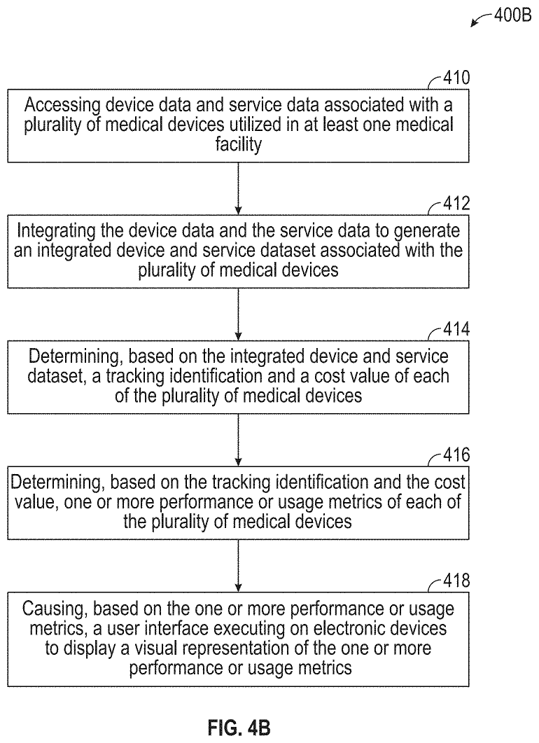

FIG. 4A illustrates a flow diagram of a method 400A for estimating performance or usage metrics of medical devices utilized within a medical facility, in accordance with the presently disclosed embodiments. The method 300 may be performed utilizing one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), or any other processing device(s) that may be suitable for processing user data or medical data), software (e.g., instructions running/executing on one or more processors), firmware (e.g., microcode), or some combination thereof.

The method 400A may begin at block 402 with one or more processing devices accessing, by the one or more computing devices, device data and service data associated with a plurality of medical devices utilized in at least one medical facility. The method 400A may continue at block 404 with one or more processing devices integrating, by the one or more computing devices, the device data and service data to generate an integrated device and service dataset associated with the plurality of medical devices. The method 400A may continue at block 406 with one or more processing devices determining, by the one or more computing devices, and based on the integrated device and service dataset, a tracking identification and a cost value of each of the plurality of medical devices. The method 400A may conclude at block 408 with one or more processing devices estimating, by the one or more computing devices, and based on the tracking identification and the cost value, one or more performance or usage metrics associated with each of the plurality of medical devices as utilized in the at least one medical facility.

FIG. 4B illustrates a flow diagram of a method 400B for generating and displaying a user interface for managing medical devices utilized in a medical facility, in accordance with the presently disclosed embodiments. The method 400B may be performed utilizing one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), or any other processing device(s) that may be suitable for processing user data or medical data), software (e.g., instructions running/executing on one or more processors), firmware (e.g., microcode), or some combination thereof.

The method 400B may begin at block 402 with one or more processing devices accessing, by the one or more computing devices, device data and service data associated with a plurality of medical devices utilized in at least one medical facility. The method 400B may continue at block 404 with one or more processing devices integrating, by the one or more computing devices, the device data and service data to generate an integrated device and service dataset associated with the plurality of medical devices. The method 400B may continue at block 406 with one or more processing devices determining, by the one or more computing devices, and based on the integrated device and service dataset, a tracking identification and a cost value of each of the plurality of medical devices. The method 400B may continue at block 408 with one or more processing devices determining, by the one or more computing devices, and based on the tracking identification and the cost value, one or more performance or usage metrics of each of the plurality of medical devices. The method 400B may conclude at block 410 with one or more processing devices causing, by the one or more computing devices, and based on the one or more performance or usage metrics, a user interface (UI) executing on one or more electronic devices to display a visual representation of the one or more performance or usage metrics.

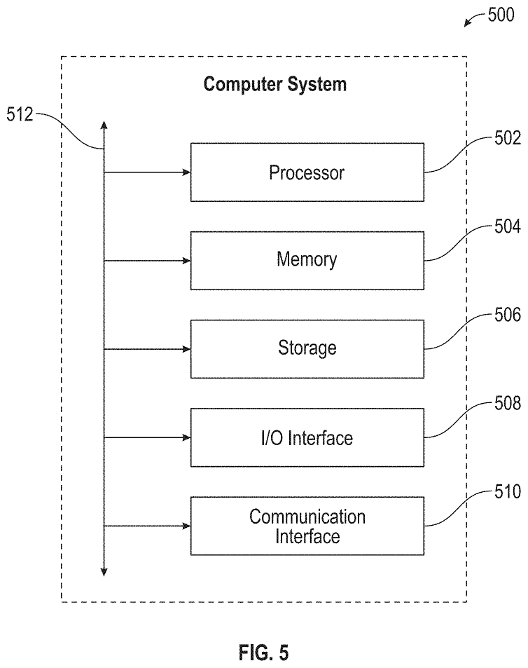

FIG. 5 illustrates an example financial services computing system 500 (which may be included as part of the financial services platform 112) that may be utilized for dynamically guiding users to request valid payment transfers, in accordance with the presently disclosed embodiments. In certain embodiments, one or more financial services computing system 500 perform one or more steps of one or more methods described or illustrated herein. In certain embodiments, one or more financial services computing system 500 provide functionality described or illustrated herein. In certain embodiments, software running on one or more financial services computing system 500 performs one or more steps of one or more methods described or illustrated herein or provides functionality described or illustrated herein. Certain embodiments include one or more portions of one or more financial services computing system 500. Herein, reference to a computer system may encompass a computing device, and vice versa, where appropriate. Moreover, reference to a computer system may encompass one or more computer systems, where appropriate.

This disclosure contemplates any suitable number of financial services computing systems 500. This disclosure contemplates computer system 500 taking any suitable physical form. As example and not by way of limitation, computer system 500 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 500 may include one or more financial services computing system 500; be unitary or distributed; span multiple locations; span multiple machines; span multiple data centers; or reside in a cloud, which may include one or more cloud components in one or more networks.

Where appropriate, one or more financial services computing system 500 may perform without substantial spatial or temporal limitation one or more steps of one or more methods described or illustrated herein. As an example, and not by way of limitation, one or more financial services computing system 500 may perform in real time or in batch mode one or more steps of one or more methods described or illustrated herein. One or more financial services computing system 500 may perform at different times or at different locations one or more steps of one or more methods described or illustrated herein, where appropriate.

In certain embodiments, computer system 500 includes a processor 502, memory 504, storage 506, an input/output (I/O) interface 508, a communication interface 510, and a bus 512. Although this disclosure describes and illustrates a particular computer system having a particular number of particular components in a particular arrangement, this disclosure contemplates any suitable computer system having any suitable number of any suitable components in any suitable arrangement. In certain embodiments, processor 502 includes hardware for executing instructions, such as those making up a computer program. As an example, and not by way of limitation, to execute instructions, processor 502 may retrieve (or fetch) the instructions from an internal register, an internal cache, memory 504, or storage 506; decode and execute them; and then write one or more results to an internal register, an internal cache, memory 504, or storage 506. In certain embodiments, processor 502 may include one or more internal caches for data, instructions, or addresses. This disclosure contemplates processor 502 including any suitable number of any suitable internal caches, where appropriate. As an example, and not by way of limitation, processor 502 may include one or more instruction caches, one or more data caches, and one or more translation lookaside buffers (TLBs). Instructions in the instruction caches may be copies of instructions in memory 504 or storage 506, and the instruction caches may speed up retrieval of those instructions by processor 502.

Data in the data caches may be copies of data in memory 504 or storage 506 for instructions executing at processor 502 to operate on; the results of previous instructions executed at processor 502 for access by subsequent instructions executing at processor 502 or for writing to memory 504 or storage 506; or other suitable data. The data caches may speed up read or write operations by processor 502. The TLBs may speed up virtual-address translation for processor 502. In certain embodiments, processor 502 may include one or more internal registers for data, instructions, or addresses. This disclosure contemplates processor 502 including any suitable number of any suitable internal registers, where appropriate. Where appropriate, processor 502 may include one or more arithmetic logic units (ALUs); be a multi-core processor; or include one or more processors 502. Although this disclosure describes and illustrates a particular processor, this disclosure contemplates any suitable processor.

In certain embodiments, memory 504 includes main memory for storing instructions for processor 502 to execute or data for processor 502 to operate on. As an example, and not by way of limitation, computer system 500 may load instructions from storage 506 or another source (such as, for example, another computer system 500) to memory 504. Processor 502 may then load the instructions from memory 504 to an internal register or internal cache. To execute the instructions, processor 502 may retrieve the instructions from the internal register or internal cache and decode them. During or after execution of the instructions, processor 502 may write one or more results (which may be intermediate or final results) to the internal register or internal cache. Processor 502 may then write one or more of those results to memory 504. In certain embodiments, processor 502 executes only instructions in one or more internal registers or internal caches or in memory 504 (as opposed to storage 506 or elsewhere) and operates only on data in one or more internal registers or internal caches or in memory 504 (as opposed to storage 506 or elsewhere).

One or more memory buses (which may each include an address bus and a data bus) may couple processor 502 to memory 504. Bus 512 may include one or more memory buses, as described below. In certain embodiments, one or more memory management units (MMUs) reside between processor 502 and memory 504 and facilitate accesses to memory 504 requested by processor 502. In certain embodiments, memory 504 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 504 may include one or more memory devices 504, where appropriate. Although this disclosure describes and illustrates particular memory, this disclosure contemplates any suitable memory.

In certain embodiments, storage 506 includes mass storage for data or instructions. As an example, and not by way of limitation, storage 506 may include a hard disk drive (HDD), a floppy disk drive, flash memory, an optical disc, a magneto-optical disc, magnetic tape, or a Universal Serial Bus (USB) drive or a combination of two or more of these. Storage 506 may include removable or non-removable (or fixed) media, where appropriate. Storage 506 may be internal or external to computer system 500, where appropriate. In certain embodiments, storage 506 is non-volatile, solid-state memory. In certain embodiments, storage 506 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 506 taking any suitable physical form. Storage 506 may include one or more storage control units facilitating communication between processor 502 and storage 506, where appropriate. Where appropriate, storage 506 may include one or more storages 506. Although this disclosure describes and illustrates particular storage, this disclosure contemplates any suitable storage.

In certain embodiments, I/O interface 508 includes hardware, software, or both, providing one or more interfaces for communication between computer system 500 and one or more I/O devices. Computer system 500 may include one or more of these I/O devices, where appropriate. One or more of these I/O devices may enable communication between a person and computer system 500. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 506 for them. Where appropriate, I/O interface 508 may include one or more device or software drivers enabling processor 502 to drive one or more of these I/O devices. I/O interface 508 may include one or more I/O interfaces 506, where appropriate. Although this disclosure describes and illustrates a particular I/O interface, this disclosure contemplates any suitable I/O interface.

In certain embodiments, communication interface 510 includes hardware, software, or both providing one or more interfaces for communication (such as, for example, packet-based communication) between computer system 500 and one or more other computer systems 500 or one or more networks. As an example, and not by way of limitation, communication interface 510 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 510 for it.

As an example, and not by way of limitation, computer system 500 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 500 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 500 may include any suitable communication interface 510 for any of these networks, where appropriate. Communication interface 510 may include one or more communication interfaces 510, where appropriate. Although this disclosure describes and illustrates a particular communication interface, this disclosure contemplates any suitable communication interface.

In certain embodiments, bus 512 includes hardware, software, or both coupling components of computer system 500 to each other. As an example, and not by way of limitation, bus 512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 512 may include one or more buses 512, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

What is claimed is:

1. A method for generating and displaying a user interface for managing medical devices utilized in a medical facility, comprising, by one or more computing devices of an equipment utilization and healthcare technology management platform:
accessing, by the one or more computing devices, device data and service data associated with a plurality of medical devices utilized in at least one medical facility;
integrating, by the one or more computing devices, the device data and service data to generate an integrated device and service dataset associated with the plurality of medical devices;
determining, by the one or more computing devices, and based on the integrated device and service dataset, a tracking identification and a cost value of each of the plurality of medical devices;
determining, by the one or more computing devices, and based on the tracking identification and the cost value, one or more performance or usage metrics of each of the plurality of medical devices; and
causing, by the one or more computing devices, and based on the one or more performance or usage metrics, a user interface (UI) executing on one or more electronic devices utilized in the at least one medical facility to display a visual representation of the one or more performance or usage metrics.

2. The method of claim 1, wherein the visual representation of the one or more performance or usage metrics comprises a real-time or near real-time dashboard associated with the plurality of medical devices utilized in the at least one medical facility.

3. The method of claim 1, wherein the device data comprises device identification data obtained from a manufacturer of each of the plurality of medical devices or a regulatory agency associated with each of the plurality of medical devices.

4. The method of claim 1, wherein the service data comprises transaction data obtained from one or more of a purchase contract associated with each of the plurality of medical devices, a service contract associated with each of the plurality of medical devices, or an enterprise resource planning (ERP) system associated with the plurality of medical devices.

5. The method of claim 1, wherein the one or more performance or usage metrics comprises one or more of a utilization metric, a location metric, a capacity metric, a status metric, an asset aging metric, a cost basis metric, a maintenance cost metric, a maintenance utilization metric, a maintenance performance metric, a vendor performance metric, a clinical unit statistic metric, or a device performance metric.

6. The method of claim 1, wherein estimating the one or more performance or usage metrics comprises estimating, by the one or more computing devices, an asset utilization of each of the plurality of medical devices or a total cost of ownership of each of the plurality of medical devices.

7. The method of claim 1, further comprising:
receiving, by the one or more computing devices, one or more user inputs from one or more of the plurality of medical devices, the one or more user inputs being performed on the one or more of the plurality of medical devices by a clinician in the at least one medical facility; and
updating, by the one or more computing devices, the UI based on the received one or more user inputs.

8. A non-transitory computer-readable medium comprising instructions that, when executed by one or more computing devices of an equipment utilization and healthcare technology management platform, cause the one or more computing devices to:
access device data and service data associated with a plurality of medical devices utilized in at least one medical facility;
integrate the device data and service data to generate an integrated device and service dataset associated with the plurality of medical devices;
determine, based on the integrated device and service dataset, a tracking identification and a cost value of each of the plurality of medical devices;
determine, based on the tracking identification and the cost value, one or more performance or usage metrics of each of the plurality of medical devices; and
cause, based on the one or more performance or usage metrics, a user interface (UI) executing on one or more electronic devices utilized in the at least one medical facility to display a visual representation of the one or more performance or usage metrics.

9. The non-transitory computer-readable medium of claim 8, wherein the visual representation of the one or more performance or usage metrics comprises a real-time or near real-time dashboard associated with the plurality of medical devices utilized in the at least one medical facility.

10. The non-transitory computer-readable medium of claim 8, wherein the device data comprises device identification data obtained from a manufacturer of each of the plurality of medical devices or a regulatory agency associated with each of the plurality of medical devices.

11. The non-transitory computer-readable medium of claim 8, wherein the service data comprises transaction data obtained from one or more of a purchase contract associated with each of the plurality of medical devices, a service contract associated with each of the plurality of medical devices, or an enterprise resource planning (ERP) system associated with the plurality of medical devices.

12. The non-transitory computer-readable medium of claim 8, wherein the one or more performance or usage metrics comprises one or more of a utilization metric, a location metric, a capacity metric, a status metric, an asset aging metric, a cost basis metric, a maintenance cost metric, a maintenance utilization metric, a maintenance performance metric, a vendor performance metric, a clinical unit statistic metric, or a device performance metric.

13. The non-transitory computer-readable medium of claim 8, wherein the instructions to estimate the one or more performance or usage metrics further comprise instructions to estimate an asset utilization of each of the plurality of medical devices or a total cost of ownership of each of the plurality of medical devices.

14. The non-transitory computer-readable medium of claim 8, wherein the instructions further comprise instructions to:
receive one or more user inputs from one or more of the plurality of medical devices, the one or more user inputs being performed on the one or more of the plurality of medical devices by a clinician in at least one medical facility; and
update the UI based on the one or more user inputs.

15. A system for generating and displaying a user interface for managing medical devices utilized in a medical facility, comprising:
one or more non-transitory computer-readable storage media including instructions; and
one or more processors coupled to the one or more storage media, the one or more processors configured to execute the instructions to:
access device data and service data associated with a plurality of medical devices utilized in at least one medical facility;
integrate the device data and service data to generate an integrated device and service dataset associated with the plurality of medical devices;
determine, based on the integrated device and service dataset, a tracking identification and a cost value of each of the plurality of medical devices;
determine, based on the tracking identification and the cost value, one or more performance or usage metrics of each of the plurality of medical devices; and
cause, based on the one or more performance or usage metrics, a user interface (UI) executing on one or more electronic devices utilized in the at least one medical facility to display a visual representation of the one or more performance or usage metrics.

16. The system of claim 15, wherein the visual representation of the one or more performance or usage metrics comprises a real-time or near real-time dashboard associated with the plurality of medical devices utilized in the at least one medical facility.

17. The system of claim 15, wherein the device data comprises device identification data obtained from a manufacturer of each of the plurality of medical devices or a regulatory agency associated with each of the plurality of medical devices.

18. The system of claim 15, wherein the service data comprises transaction data obtained from one or more of a purchase contract associated with each of the plurality of medical devices, a service contract associated with each of the plurality of medical devices, or an enterprise resource planning (ERP) system associated with the plurality of medical devices.

19. The system of claim 15, wherein the one or more performance or usage metrics comprises one or more of a utilization metric, a location metric, a capacity metric, a status metric, an asset aging metric, a cost basis metric, a maintenance cost metric, a maintenance utilization metric, a maintenance performance metric, a vendor performance metric, a clinical unit statistic metric, or a device performance metric.

20. The system of claim 15, wherein the instructions to estimate the one or more performance or usage metrics further comprise instructions to estimate an asset utilization of each of the plurality of medical devices or a total cost of ownership of each of the plurality of medical devices.
